(19) United States
(12) Patent Application Publication
(10) Pub. No.: US 2023/0010903 A1
(43) Pub. Date: Jan. 12, 2023

(54) LOCKING IRRIGATION VALVE

(71) Applicant: Prodeon Medical Corporation, Taipei (TW)

(72) Inventors: Jimmy Jen, Saratoga, CA (US); Kenneth Chih-Ping Chang, San Jose, CA (US); Gary Daniel Zaretzka, Sunnyvale, CA (US); Po-Hua Lee, Sunnyvale, CA (US)

(73) Assignee: Prodeon Medical Corporation, Taipei (TW)

(21) Appl. No.: 17/858,606

(22) Filed: Jul. 6, 2022

Related U.S. Application Data
(60) Provisional application No. 63/219,092, filed on Jul. 7, 2021.

(51) Int. Cl. A61B 1/015

(52) U.S. Cl. CPC A61B 1/015

(57) ABSTRACT

A locking irrigation valve with is disclosed that features a valve body having a lumen, an irrigation port in fluid communication with the lumen, a connector configured couple to a cystoscope so that the lumen is aligned with a working channel of the cystoscope, a collet having a compressible inner bore and a lock configured to engage the collet and compress the inner bore.

### RELATED APPLICATIONS

This application claims priority to U.S. Provisional Application No. 63/219,092, filed Jul. 7, 2021. The priority of this application is expressly claimed, and the disclosure is hereby incorporated by reference in its entirety.

### FIELD OF THE PRESENT DISCLOSURE

This disclosure relates to devices for managing or treating body tissues obstructing a hollow body lumen, such as the prostatic lobe tissues obstructing the urethra and more particularly, to devices and methods for facilitating the use of cystoscopes for diagnostic and therapeutic applications.

### BACKGROUND

The prostate is a walnut-shaped gland that wraps around the urethra through which urine is expelled from the bladder and plays a crucial role in the reproductive system of men. Although the gland starts out small, it tends to enlarge as a man ages. An excessively enlarged prostate results in a disease known as benign prostatic hyperplasia (BPH). Benign prostatic hyperplasia (BPH) refers to the abnormal, but non-malignant (non-cancerous) growth of the prostate observed very commonly in aging men. BPH is a chronic condition and is associated with the development of urinary outflow obstruction or luminal narrowing in the prostatic urethra. Bladder outlet obstruction (BOO) refers to a blockage at the base of the bladder that reduces or stops the flow of urine into the urethra and may be secondary to BPH. A range of related disorders referred to collectively as Lower Urinary Tract Symptoms (LUTS) can result, including sexual dysfunction, frequent urination, difficulty in voiding urine, urinary retention, urinary leakage, and urinary tract and bladder infections that worsen as the abnormal growth in the prostate enlarges and progresses.

Cystoscopes are an important tool for use in diagnostic or therapeutic procedures related to conditions such as those noted above, whether surgical or less invasive techniques include implanting a device within the prostatic urethra that is designed to increase the diameter of the urethra. Accordingly, cystoscopes are used to examine the urinary tract and often times perform simple procedures to diagnose and correct urinary tract issues. They are composed of a handpiece with a light source and either a fiberoptic/lens for direct viewing or a system that contains or connects to a camera at the end of the device. Cystoscopes may be rigid or flexible, with the flexible versions offering more patient comfort by allowing steering of the tip via a control on the handle. Cystoscopes also often include irrigation functionality, so that delivery of saline or other suitable fluid through a working channel facilitates viewing and imaging by clearing away debris/blood from the camera's view. Procedures may be performed with the cystoscope by advancing elongated tools or instruments through the working channel. The tools can be simple graspers or baskets or more complex device delivery and retrieval tools but generally must fit through the irrigation port channel.

It would be beneficial to provide devices and methods for firmly securing a grasper or other medical instruments or devices introduced through a valve used in endourology and endovascular procedures. Current valves, such as a rotating hemostasis valve used in endovascular procedures and irrigation valves used in endourology procedures are unable to firmly hold the devices introduced through the lumen in place. For example, it is desirable to fix the urological instrument's (such as a grasper's) position relative to the cystoscope during foreign body retrieval from the urinary tract. The length of the grasper is typically longer than the working channel of the cystoscope. During the retrieval procedure, after the user has secured the foreign body with grasper, the cystoscope and grasper are usually removed from the urethra, as a system, at the same time. Due to the extended length of the grasper, there is a possibility that the cystoscope could be removed the urethra much earlier than the grasper. The user could lose visualization of the foreign body during retrieval and jeopardize patient safety. There is also the possibility of losing (or embolizing) the foreign body during the retrieval procedure. Correspondingly, it would be desirable to provide a locking irrigation valve that firmly secures a grasper or other medical instruments or devices, such that the valve remains capable of supplying irrigation fluid. The present disclosure satisfies these and other needs as detailed in the specification and drawings.

### SUMMARY

This disclosure is directed to a locking irrigation valve with a valve body having a lumen, an irrigation port in fluid communication with the lumen, a connector configured couple to a cystoscope so that the lumen is aligned with a working channel of the cystoscope, a collet having a compressible inner bore and a lock configured to engage the collet and compress the inner bore.

In one aspect, the lock may be actuated by a lever.

In one aspect, the collet may have a tapered exterior profile and wherein a cap having a complimentary internal wedge is disposed over collet such that actuation of the lever results in an axial movement of the cap that compresses the inner bore. The cap may be driven by pins that engage cammed slots in the lever.

In one aspect, the collet may have a textured inner surface configured to increase friction when compressed.

In one aspect, the lever may have a plurality of discrete clamping positions.

In one aspect, the lever may have a seal to inhibit leakage of irrigation fluid from the lumen of the valve body into the collet.

In one aspect, the collet may have a plurality of deflectable arms configured to be engaged by actuation of the lever. An inner profile of the lever may be configured to engage the deflectable arms when the lever is rotated. The inner profile may feature lobes within recesses of the inner profile.

This disclosure also includes a method for selectively securing an elongated device advanced through a valve into a working channel of a cystoscope. The method may involve providing a locking irrigation valve having a valve body with a lumen and a collet having a compressible inner bore aligned with the lumen, coupling the locking irrigation valve to the cystoscope so that the lumen is aligned with the working channel of the cystoscope, advancing the elongated device through the inner bore and engaging the collet with a lock to compress the inner bore and secure the elongated device.

In one aspect, engaging the collet may include actuating a lever.

In one aspect, actuating the lever may cause axial movement of a cap disposed over the collet, wherein the collet has a tapered exterior profile and wherein the cap has a complimentary internal wedge, to compress the inner bore.

In one aspect, actuating the lever may cause an inner profile of the lever to engage arms of the collet to deflect them inwards when the lever is rotated to compress the inner bore.

In one aspect, fluid may be introduced through the irrigation port before or after engaging the collet with the lock.

In one aspect, the elongated device may be a grasper. The method may therefore include using the grasper to secure a foreign body before engaging the collet with the lock and subsequently removing the foreign body by manipulating the cystoscope and grasper as a single unit.

### BRIEF DESCRIPTION OF THE DRAWINGS

Further features and advantages will become apparent from the following and more particular description of the preferred embodiments of the disclosure, as illustrated in the accompanying drawings, and in which like referenced characters generally refer to the same parts or elements throughout the views, and in which:

FIG. 1 schematically depicts a side view of a locking irrigation valve according to an embodiment.

FIG. 2 schematically depicts a cross-sectional view of the valve of FIG. 1 according to an embodiment.

FIG. 3 schematically depicts an isometric view of the valve of FIG. 1 according to an embodiment.

FIG. 4 schematically depicts a side view of a locking irrigation valve having a Y arm configuration according to an embodiment.

FIG. 5 schematically depicts a side view of a detail of the collet of the valve of FIG. 4 according to an embodiment.

FIG. 6 schematically depicts an isometric view of a detail of the collet of the valve of FIG. 4 according to an embodiment.

FIG. 7 schematically depicts a side view of a detail of the collet of the valve of FIG. 4 with the locking arm according to an embodiment.

### DETAILED DESCRIPTION

At the outset, it is to be understood that this disclosure is not limited to particularly exemplified materials, architectures, routines, methods or structures as such may vary. Thus, although a number of such options, similar or equivalent to those described herein, can be used in the practice or embodiments of this disclosure, the preferred materials and methods are described herein.

It is also to be understood that the terminology used herein is for the purpose of describing particular embodiments of this disclosure only and is not intended to be limiting.

The detailed description set forth below in connection with the appended drawings is intended as a description of exemplary embodiments of the present disclosure and is not intended to represent the only exemplary embodiments in which the present disclosure can be practiced. The term “exemplary” used throughout this description means “serving as an example, instance, or illustration,” and should not necessarily be construed as preferred or advantageous over other exemplary embodiments. The detailed description includes specific details for the purpose of providing a thorough understanding of the exemplary embodiments of the specification. It will be apparent to those skilled in the art that the exemplary embodiments of the specification may be practiced without these specific details.

For purposes of convenience and clarity only, directional terms, such as top, bottom, left, right, up, down, over, above, below, beneath, rear, back, and front, may be used with respect to the accompanying drawings. These and similar directional terms should not be construed to limit the scope of the disclosure in any manner.

Unless defined otherwise, all technical and scientific terms used herein have the same meaning as commonly understood by one having ordinary skill in the art to which the disclosure pertains. Moreover, as used in this specification and the appended claims, the singular forms “a,” “an” and “the” include plural referents unless the content clearly dictates otherwise.

With respect to orientation of the various structures and anatomical references described herein, the term “proximal” and “distal” are relative to the perspective of the medical professional, such as a urologist, who is manipulating the delivery system of the disclosure to deploy the implants described herein. Accordingly, those features of the delivery system held by the hand of the urologist are at the “proximal” end and the assembled system and the implant, initially in its compressed configuration, is located at the “distal” end of the delivery system.

As noted above, the techniques of this disclosure feature a grasper having an auto-locking function. The handle includes a self-actuating mechanism to automatically apply a grasping force to the jaws at the distal end of the device, reducing or eliminating the need for the user to apply the constant forward pressure described above that is characteristic of the prior art graspers. Particularly, a grasper embodying features of this disclosure employ the self-actuating mechanism to close and maintain the distal jaw closure.

As will be detailed in the following discussion, the techniques of this disclosure are directed to a locking irrigation valve that secures the position of an elongated device introduced into the working channel of a cystoscope. For example, the elongated device may be a grasper or another common urological instrument used during foreign body retrieval. However, it should also be appreciated that these techniques may be applied to other procedures where medical devices are introduced through valves. In the context of removal of a foreign body from the urinary tract, aspects of this disclosure provide several benefits. Notably, a locking valve pursuant to this disclosure is compatible with grasper or instrument shaft or other medical devices introduced through the valve opening. The valve also features a locking function configured to exert sufficient force for securing the elongated device at a fixed position within the working channel of the cystoscope channel during retrieval of the foreign body while having the capability of being unlocked at any time by the user. Moreover, the valve allows irrigation or infusion of fluids, as needed, through the valve and cystoscope instrument channel. The locking irrigation valve also functions as a regular valve when the grasper or instrument is not present.

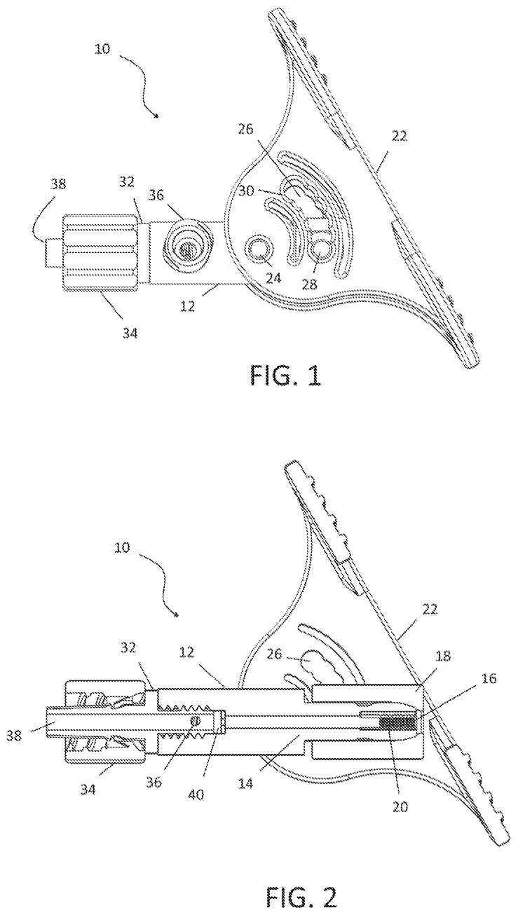

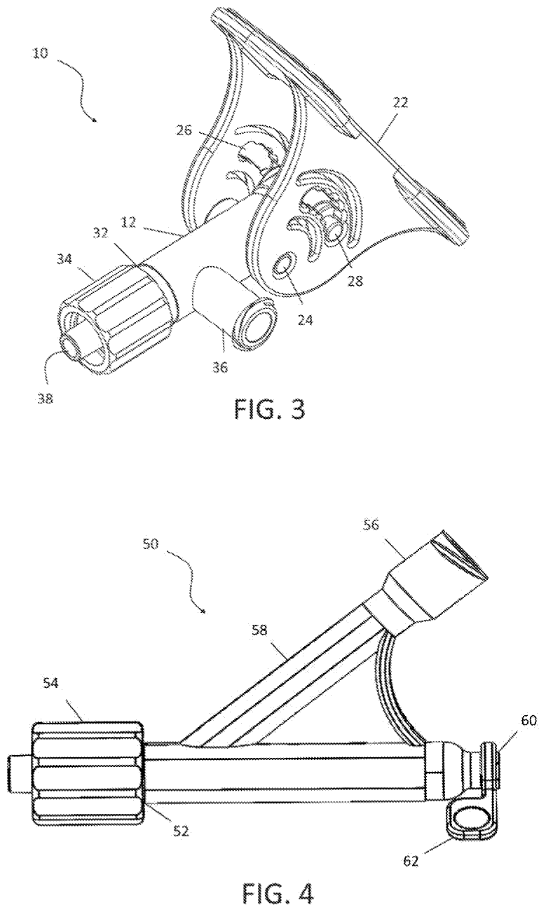

To help illustrate aspects of this disclosure, side, cross-sectional and isometric views of locking irrigation valve 10 are shown in FIGS. 1-3, respectively. A body 12 of valve 10 features a collet 14 at the proximal end formed by a plurality of longitudinal kerfs and having an external profile that is tapered in the kerfed portion. As detailed below, an inner bore 16 of collet 14 is compressible from its nominal diameter to provide a gripping or clamping force on an instrument deployed through valve 10. Correspondingly, a collet cap 18 has a complimentary internal wedge profile such that axial movement of cap 18 along the longitudinal axis of body 12 compresses collet 14, decreasing its inner diameter. As desired, the inner surfaces of collet 14 may feature a ridged or otherwise textured profile 20 to enhance the engagement with the shaft of an elongated device advanced through collet 14. A locking lever 22 is provided at the proximal end of valve 10 that pivots on axle 24. Opposing portions of lever 22 have cam slots 26 that receive pins 28 projecting from cap 16. As will be appreciated, cam slots 26 may be configured to urge pins 28 distally as lever 22 is pivoted from an open configuration to a closed configuration. Accordingly, cap 18 is driven distally so that the complementary tapers of collet 14 and cap 18 compress collet 14 as discussed above to generate the desired clamping force within bore 16 and lock an elongated medical device positioned within the bore. As shown, cam slots 26 may feature detents 30 along the range of travel of pins 28 to maintain lever 22 in different, discrete positions that correspond to different degrees of clamping force.

A Luer connector 32 having a locking ring 34 is employed as known in the art to allow coupling to a respective connector aligned with the working channel of a cystoscope. An irrigation port 36 is in fluid communication with a lumen 38. Lumen 38 is aligned with inner bore 16 of collet 14 so that the shaft of an elongated device may be advanced sequentially through collet 14 and lumen 38 into the working channel of a cystoscope. Correspondingly, irrigation fluid may be introduced through port 36 and into the working channel of the cystoscope at any time during the procedure, regardless of the state of collet 14. An external taper on Luer connector 32 inhibits fluid leakage from lumen 38 at the cystoscope junction and a seal 40 similarly inhibits fluid leakage proximally from collet 14.

In an exemplary method, Luer connector 32 may be used to secure valve 10 to the cystoscope by engaging locking ring 34. The user may then connect an irrigation line to port 36 and advance a grasper or other suitable elongated medical device through collet 14 and lumen 38 into the working channel of the cystoscope. Again in the context of foreign body retrieval, the grasper may be used to grip and secure the foreign body in preparation for removal. The user then pivots lever 22 to compress collet 14 and lock it on to the shaft of the grasper or other elongated device. The cystoscope, valve 10 and grasper or other device may then be withdrawn as a single unit to accomplish removal of the foreign body. At any time during or after the procedure, lever 22 may be pivoted in the opposite direction so that cam slots 2 engage pins 28 to drive cap 18 proximally, allowing collet 14 to return to its nominal diameter and release the grasper or other device.

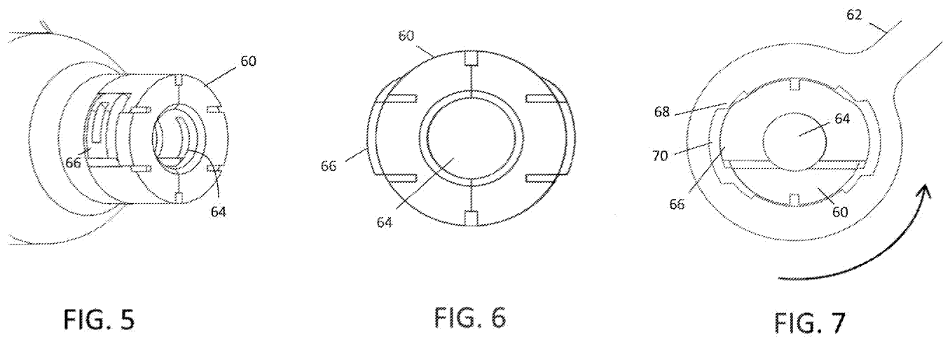

As another exemplary illustration of the techniques of this disclosure, locking irrigation valve 50 is shown in FIG. 4 as an alternative embodiment. Here, valve 50 is configured as a standard hemostasis Y-connector body, with a Luer connector 52 and locking ring 54 at its distal end and an irrigation port 56 on side arm 58, all with similar functionality to that described above. The proximal end of valve 50 has a collet 60 with a locking lever 62 that may be used to engage the shaft of a grasper or other elongated medical device in a manner similar to that described above. FIGS. 5 and 6 show detail isometric and end views, respectively, of collet 60 with lever 62 removed for clarity. Notably, without engagement of lever 62, collet 60 has a nominal diameter inner bore 64. Two or more arms 66 are provided around bore 64. Arms 66 are deflectable and are biased outwards but have inward mobility so that rotation of lever 62 causes arms 66 to push into the nominal diameter of bore 64 to compress it and create sufficient friction to lock the grasper shaft or other elongated medical device to valve 50. With specific reference to FIG. 7, lever 62 is depicted as encircling collet 60 and has an inner profile configure to engage arms 66, in this embodiment shown as lobes 68 formed within recesses 70. Accordingly, when lever 62 is rotated such that recesses 70 are positioned over arms 66, bore 64 has its nominal diameter and when lever 62 is rotated so that lobes 66 engage arms 66, they are driven inwards to generate the noted clamping force by compressing the inner bore. Once more, this has the result of coupling the grasper or other instrument to the cystoscope so they move together.

As will be appreciated from the above discussion and figures, the locking irrigation valves may be used with a grasper during the retrieval of foreign object, including a stent, expander or other implant, or a naturally occurring deposit such as a stone or calculi, from the prostatic urethra, urinary bladder, ureters, kidneys urinary tract or urinary system in addition to other suitable applications. By employing the techniques of this disclosure, such a valve may exhibit a number of beneficial characteristics, including without limitation:
being compatible with grasper or instrument shaft or other medical devices introduced through the valve opening;
being capable of locking on to the grasper or instrument shaft with sufficient force to retrieve the foreign body;
being unlockable at any time by the user;
allowing irrigation or infusion of fluids, as needed, through the valve and scope instrument channel;
being capable of securing to the cystoscope instrument or working channel; and
functioning as a regular valve when the grasper or instrument is not present.

The exemplary embodiments disclosed above are merely intended to illustrate the various utilities of this disclosure. It is understood that numerous modifications, variations and combinations of functional elements and features of the present disclosure are possible in light of the above teachings and, therefore, within the scope of the appended claims, the present disclosure may be practiced otherwise than as particularly disclosed and the principles of this disclosure can be extended easily with appropriate modifications to other applications.

All patents and publications are herein incorporated for reference to the same extent as if each individual publication was specifically and individually indicated to be incorporated by reference. It should be understood that although the present disclosure has been specifically disclosed by preferred embodiments and optional features, modification and variation of the concepts herein disclosed may be resorted by those skilled in the art, and that such modifications and variations are considered to be within the scope of this disclosure.

What is claimed is:

1. A locking irrigation valve, comprising:
a valve body having a lumen;
an irrigation port in fluid communication with the lumen;
a connector configured couple to a cystoscope so that the lumen is aligned with a working channel of the cystoscope;
a collet having a compressible inner bore; and
a lock configured to engage the collet and compress the inner bore.

2. The valve of claim 1, wherein the lock is actuated by a lever.

3. The valve of claim 2, wherein the collet has a tapered exterior profile and wherein a cap having a complimentary internal wedge is disposed over collet such that actuation of the lever results in an axial movement of the cap that compresses the inner bore.

4. The valve of claim 3, wherein the cap is driven by pins that engage cammed slots in the lever.

5. The valve of claim 2, wherein the collet has a textured inner surface configured to increase friction when compressed.

6. The valve of claim 2, wherein the lever has a plurality of discrete clamping positions.

7. The valve of claim 1, wherein the lever has a seal to inhibit leakage of irrigation fluid from the lumen of the valve body into the collet.

8. The valve of claim 2, wherein the collet comprises a plurality of deflectable arms configured to be engaged by actuation of the lever, such.

9. The valve of claim 8, wherein an inner profile of the lever is configured to engage the deflectable arms when the lever is rotated.

10. The valve of claim 9, wherein the inner profile comprises lobes within recesses of the inner profile.

11. A method for selectively securing an elongated device advanced through a valve into a working channel of a cystoscope, comprising:
providing a locking irrigation valve having a valve body with a lumen and a collet having a compressible inner bore aligned with the lumen,
coupling the locking irrigation valve to the cystoscope so that the lumen is aligned with the working channel of the cystoscope;
advancing the elongated device through the inner bore; and
engaging the collet with a lock to compress the inner bore and secure the elongated device.

12. The method of claim 11, wherein engaging the collet comprises actuating a lever.

13. The method of claim 12, wherein actuating the lever causes axial movement of a cap disposed over the collet, wherein the collet has a tapered exterior profile and wherein the cap has a complimentary internal wedge, to compress the inner bore.

14. The method of claim 12, wherein actuating the lever causes an inner profile of the lever to engage arms of the collet to deflect them inwards when the lever is rotated to compress the inner bore.

15. The method of claim 11, further comprising introducing fluid through the irrigation port before or after engaging the collet with the lock.

16. The method of claim 11, wherein the elongated device is a grasper.

17. The method of claim 16, further comprising using the grasper to secure a foreign body before engaging the collet with the lock and subsequently removing the foreign body by manipulating the cystoscope and grasper as a single unit.
